(19) United States
(12) Patent Application Publication
(10) Pub. No.: US 2015/0153472 A1
(43) Pub. Date: Jun. 4, 2015

(54) Detecting Hydrocarbons in a Geological Structure

(71) Applicant: WILLIAM MARSH RICE UNIVERSITY, Houston, TX (US)

(72) Inventors: James M. Tour, Bellaire, TX (US); Wei Lu, Houston, TX (US); Chih-Chau Hwang, Houston, TX (US)

(73) Assignee: William Marsh Rice University, Houston, TX (US)

(21) Appl. No.: 14/408,917

(22) PCT Filed: Jun. 24, 2013
(86) PCT No.: PCT/US2013/047425

§ 371 Date: Dec. 17, 2014

Related U.S. Application Data
(60) Provisional application No. 61/681,743, filed on Aug. 10, 2012.
(60) Provisional application No. 61/663,134, filed on Jun. 22, 2012.

(51) Int. Cl. G01V 3/26; G01N 33/24

(52) U.S. Cl. CPC G01V 3/26; G01N 33/241

(57) ABSTRACT

Magnetic nanoparticles are utilized for magnetically detecting hydrocarbons in a geological structure. The magnetic nanoparticles generally include a core particle and a temperature responsive polymer associated with the core particle. The temperature responsive polymer may include polyacrylamides, polyethylene glycols, or combinations thereof. The temperature responsive polymer facilitates an agglomeration of the nanoparticles in a fluid at an organic/aqueous interface of the fluid, an organic phase of the fluid, or combinations thereof. The agglomeration may occur at a specific temperature or temperature range.

### CROSS-REFERENCE TO RELATED APPLICATIONS

This application claims priority to U.S. Provisional Patent Application No. 61/663,134, filed on Jun. 22, 2012; and U.S. Provisional Patent Application No. 61/681,743, filed on Aug. 10, 2012. The entirety of each of the aforementioned applications is incorporated herein by reference.

### STATEMENT REGARDING FEDERALLY SPONSORED RESEARCH

Not applicable.

### BACKGROUND

Current systems and methods to detect hydrocarbons in geological structures have numerous limitations in terms of sensitivity and selectivity. Therefore, more effective systems and methods are desired for detecting hydrocarbons in geological structures.

### SUMMARY

The present disclosure generally pertains to magnetic nanoparticles for magnetically detecting hydrocarbons in a geological structure. Embodiments of the present disclosure pertain to methods for magnetically detecting hydrocarbons in a geological structure. In some embodiments, such methods comprise: injecting magnetic nanoparticles of the present disclosure into the geological structure; generating or enhancing a magnetic field in the geological structure; detecting a magnetic signal; and correlating the detected magnetic signal to location of hydrocarbons in the geological structure. In some embodiments, the geological structure is an oil or gas reservoir. In some embodiments, the hydrocarbons are crude oil. In some embodiments, the magnetic nanoparticles in contact with hydrocarbons are illuminated as a result of the generated or enhanced magnetic field. In some embodiments, the magnetic nanoparticles generally include: a core particle; and a temperature responsive polymer associated with the core particle. In some embodiments, the temperature responsive polymer is selected from the group consisting of polyacrylamides, polyalcohols, polyethylene glycols, and combinations thereof. In some embodiments, the temperature responsive polymer facilitates an agglomeration of the magnetic nanoparticles in a fluid at an organic/aqueous interface of the fluid, an organic phase of the fluid, or combinations thereof. In some embodiments, the agglomeration occurs at a specific temperature or temperature range. In some embodiments, the core particle comprises oxidized carbon black. In some embodiments, the core particle is a carbon-coated magnetite nanoparticle. In some embodiments, the temperature responsive polymer is covalently associated with the core particle. In some embodiments, the temperature responsive polymer is selected from the group consisting of poly(N-isopropylacrylamide), N-isopropylacrylamide, polyethylene-b-poly(ethylene glycol), and combinations thereof. In some embodiments, the nanoparticles of the present disclosure may also be associated with amphiphilic polymers, hydrophilic polymers, hydrophobic polymers, and combinations thereof.

### BRIEF DESCRIPTION OF THE FIGURES

FIG. 1 provides an illustration of a temperature responsive magnetic nanoparticle.

FIG. 2 provides a scheme for detecting hydrocarbons in geological structures through the use of temperature responsive magnetic nanoparticles.

FIGS. 3A-3B provide diagrams illustrating schemes for magnetically detecting hydrocarbons in a geological structure through the use of temperature responsive magnetic nanoparticles. FIG. 3A shows a scheme where control magnetic nanoparticles stay in the aqueous phase of fluids in the geological structure. FIG. 3B shows a scheme where temperature responsive magnetic nanoparticles migrate to the aqueous/organic interface (i.e., oil/water interface) or even into the organic phase (i.e., oil phase) of fluids in the geological structure. The residual oil domains in the porous rocks can be constructed by comparing the magnetic resonance images generated in FIGS. 3A-3B. FIG. 3C shows a scheme for illuminating the residual oil regions in the geological structure.

FIGS. 4A-4B provide a scheme for the preparation of various temperature responsive magnetic nanoparticles. FIG. 4A provides a scheme for the preparation of polyacid-coated magnetite nanoparticles via (1) a co-precipitation method and (2) a thermal decomposition method. The control magnetite nanoparticles could be prepared via attaching poly(vinyl alcohol)1 (PVA) through EDC coupling (3). FIG. 4B provides a scheme for the synthesis of polymer-functionalized carbon-coated magnetite nanoparticles using macro polymer initiators.

FIG. 5 shows an example of how temperature responsive magnetic nanoparticles can agglomerate at the organic/aqueous interphase of a fluid at a specific temperature. Vial (a) in FIG. 5 shows an image of poly(N-isopropylacrylamide)-functionalized oxidized carbon black nanoparticle (PNIPAM-OCB) in synthetic sea brine at room temperature. Vial (b) in FIG. 5 shows the PNIPAM-OCB nanoparticles after being heated at 80° C. for 15 minutes. The PNIPAM-OCB nanoparticles agglomerate at the aqueous/organic interface, thereby giving a high local concentration of magnetic nanoparticles at the interface.

FIGS. 6A-6C provide a scheme and images relating to the synthesis and characterization of graphene-covered metal nanoparticles (hereinafter “carbon onions”). FIG. 6A provides a scheme for the synthesis of carbon onions. FIGS. 6B-6C provide high resolution transmission electron microscopy (TEM) images of the carbon onions. TEM images at 50 nm scale (FIG. 6B) and 5 nm scale (FIG. 6C) are shown.

FIGS. 7A-7B provide data relating to the characterization of the carbon onions shown in FIG. 6. FIG. 7A shows the X-ray diffraction pattern of the carbon onions. FIG. 7B shows the magnetization measurement of the carbon onions.

FIGS. 8A-8C provide schemes for the functionalization of carbon onions. FIG. 8A provides a scheme for the functionalization of carbon onions with polyethyleneimines (PEI). FIG. 8B provides a scheme for the preparation of oxidized carbon onions. FIG. 8C provides a scheme for the preparation of sulfated and PVA-functionalized carbon onions.

FIG. 9 provides data relating to the characterization of various types of carbon onions.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise. Parameters disclosed herein (e.g., temperature, time, concentrations, etc.) may be approximate.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

As energy demand continues to increase, it is desirable to produce as much oil as possible from existing and new oil wells. After primary and secondary recovery processes in subsurface oil extraction, up to two-thirds (sometimes more) of the original oil remains trapped in the reservoirs, since the residual oil is too viscous to flow and it remains as isolated droplets in the porous media. Furthermore, since it is unknown how much oil remains downhole, the well operators do not know how much to invest in the oil removal process. Hence, many oil operators often move on to other wells. Additional water flooding cannot effectively displace all the trapped oil droplets due to the high interfacial tension or bypassing of the trapped oil.

Surfactant flooding that provides both low interfacial tension between the water and the residual oil and the viscosity required for mobility control most likely will just follow the water channels formed in the most permeable areas, thus leaving oil-containing areas untouched. Furthermore, use of surfactants is costly and only justified when the market price of oil is high.

Moreover, illumination of untouched downhole areas is crucial for an assessment to know whether there is sufficient oil remaining downhole to warrant further use of extraction methods. Therefore, improving methods to assess the extent and location of remaining oil content downhole is essential for the industry to maximize return from its existing wells. This complements the improving of existing techniques widely used in enhanced oil recovery (EOR). However, before EOR is warranted, it is beneficial and economically and environmentally prudent to have an assessment of the amount of remaining downhole oil content and its precise location in that downhole environment.

Tracers have been used to map entry and exit well correlations in the oil-field. However, many of the existing tracers do not provide any information about the environment between the entry and exit locations. Thus, new systems and methods are desired for detecting hydrocarbons in geological structures.

In some embodiments, the present disclosure pertains to nanoparticles for magnetically detecting hydrocarbons in geological structures. In some embodiments, the present disclosure pertains to systems and methods of detecting hydrocarbons in geological structures. As set forth in more detail herein, various nanoparticles may be utilized to detect hydrocarbons in various geological structures. In addition, various systems and methods may be utilized to detect the presence of hydrocarbons in geological structures.

Nanoparticles

Embodiments of the present disclosure pertain to magnetic nanoparticles for magnetically detecting hydrocarbons in various geological structures. In some embodiments, the magnetic nanoparticles generally comprise a core particle and a temperature responsive polymer associated with the core particle. In some embodiments, the core particle is also associated with an amphiphilic polymer, a hydrophilic polymer, a hydrophobic polymer, and combinations thereof.

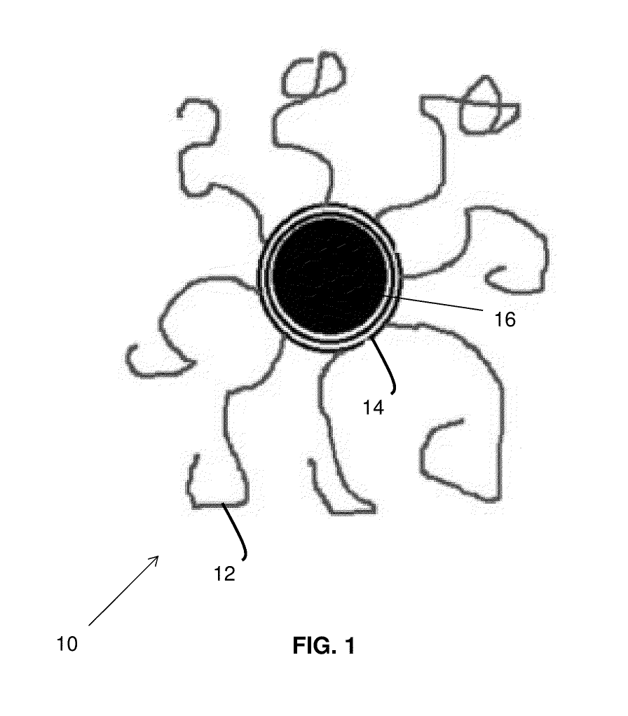

An exemplary magnetic nanoparticle is illustrated in FIG. 1. In this embodiment, magnetic nanoparticle 10 includes magnetite 16 as a core particle. In this embodiment, magnetite 16 is coated with carbon shells 14. In addition, multiple temperature responsive polymers 12 are covalently associated with carbon shell 14. As set forth in more detail herein, the nanoparticles of the present disclosure may contain various core particles that are associated with various types of temperature responsive polymers, amphiphilic polymer, hydrophilic polymers, and hydrophobic polymers.

Core Particles

Core particles generally refer to particles that can be transported through a geological structure. In some embodiments, it is desirable for the core particles to be stable to subsurface conditions. In some embodiments, it is also desirable for the core particles to endure various conditions in geological structures, such as high temperatures and salinities. In some embodiments, it is also desirable for the core particles to have mobility through different rocks in geological structures. In some embodiments, the core particles are magnetic. In some embodiments, the core particles become magnetic after becoming associated with one or more magnetic coatings.

The magnetic nanoparticles of the present disclosure may contain various core particles. In some embodiments, the core particles may include at least one of magnetite nanoparticles, metal oxide nanoparticles, iron oxide nanoparticles, mixed iron oxide and metal oxide nanoparticles, iron nanoparticles, carbon black, functionalized carbon black, oxidized carbon black, carboxyl functionalized carbon black, carbon nanotubes, functionalized carbon nanotubes, graphenes, graphene oxides, graphene nanoribbons, graphene oxide nanoribbons, metal nanoparticles, silica nanoparticles, silicon nanoparticles, silicon oxide nanoparticles, silicon nanoparticles bearing a surface oxide, and combinations thereof.

In some embodiments, the core particle is oxidized carbon black. In some embodiments, the core particle is a magnetite nanoparticle. In some embodiments, the core particle is carbon-coated. In some embodiments, the core particles may be a carbon-coated magnetite nanoparticle, such as a polyacid-coated magnetite nanoparticle, a poly(vinyl alcohol)-coated magnetite nanoparticle, a poly(vinyl sulfate) magnetite nanoparticle, a (sulfonate)-coated magnetite nanoparticle, or combinations thereof. In some embodiments, the polyacid could be an organic acid, such as citric acid, tartaric acid, or poly(acrylic acid).

In some embodiments, the core particle is a graphene-covered metal nanoparticle. In some embodiments, the graphene-covered metal nanoparticle contains a metal core that is coated with one or more graphene layers. In some embodiments, the metal core may include one or more metals. In some embodiments, the metal core includes a mixture of iron and nickel. In some embodiments, the graphene-covered metal nanoparticle may be functionalized with one or more functionalizing agents. For instance, in some embodiments, the graphene-covered metal nanoparticles may be functionalized with sulfur groups (e.g., sulfates, sulfonates, and combinations thereof), polymers (e.g., polyvinyl alcohol, polyethyleneimine, and combinations thereof), carboxyl groups, and combinations thereof.

In various embodiments, functionalized (e.g., oxidized) core particles may be prepared by reacting a dispersion of core particles with a mixture of fuming sulfuric acid and nitric acid. In more specific embodiments, oxidized carbon black may be prepared by a reaction of carbon black particles with an oxidizing agent, such as KMnO₄ in sulfuric acid or in a mixture of sulfuric acid and phosphoric acid. In some embodiments, the oxidized carbon black molecules may be highly oxidized and contain various oxidized functionalities, such as, for example, carboxylic acids, ketones, hydroxyl groups, and epoxides.

In some embodiments, the core particles of the present disclosure may be uncoated. In some embodiments, the core particles of the present disclosure may be coated with various coatings, such as polymers, surfactants, and combinations thereof.

The core particles of the present disclosure can have various sizes. For instance, in some embodiments, the core particles of the present disclosure can have diameters that range from about 1 nm to about 1 μm. In some embodiments, the core particles of the present disclosure can have diameters that range from about 1 nm to about 500 nm. In some embodiments, the core particles of the present disclosure can have diameters that are less than about 200 nm. In some embodiments, the core particles of the present disclosure can have diameters that are about 100 nm to about 200 nm. In some embodiments, the core particles of the present disclosure can have diameters that range from about 10 nm to about 50 nm. In some embodiments, the core particles of the present disclosure can have diameters that range from about 2 nm to about 200 nm.

The core particles of the present disclosure can also have various arrangements. For instance, in some embodiments, the core particles of the present disclosure may be individualized. In some embodiments, the core particles of the present disclosure may be in aggregates or clusters. In some embodiments, the core particles of the present disclosure may be in the form of clusters, where each cluster has about 3 to 5 core particles that are associated with one another.

The core particles of the present disclosure may also have various charges. For instance, in some embodiments, the core particles of the present disclosure may be positively charged. In some embodiments, the core particles of the present disclosure may be negatively charged. In some embodiments, the core particles of the present disclosure may be neutral.

Temperature Responsive Polymers

Temperature responsive polymers generally refer to polymers that facilitate an agglomeration of the nanoparticles in a fluid at an organic/aqueous interface of the fluid, an organic phase of the fluid, or combinations thereof. In some embodiments, the agglomeration occurs at a specific temperature or temperature range. In some embodiments, the temperature or temperature range in which nanoparticle agglomeration occurs may be referred to as the phase inversion temperature. In some embodiments, the phase inversion temperature may range from about 75° C. to about 150° C.

The core particles of the present disclosure may be associated with various temperature responsive polymers. In some embodiments, the temperature responsive polymer may include at least one of polyacrylamides, polyalcohols, polyethylene glycols, and combinations thereof. In some embodiments, the temperature responsive polymer may include at least one of poly(N-isopropylacrylamide) (PNIPAM), N-isopropylacrylamide, polyethylene-b-poly(ethylene glycol), and combinations thereof. In some embodiments, the temperature-responsive polymer may include copolymers of N-isopropylacrylamide and polyethylene-b-poly(ethylene glycol).

The temperature responsive polymers of the present disclosure may be associated with core particles in various manners. In some embodiments, the temperature responsive polymers of the present disclosure may be associated with core particles through non-covalent bonds, such as ionic interactions, acid-base interactions, hydrogen bonding interactions, pi-stacking interactions, van der Waals interactions, adsorption, physisorption, self-assembly, sequestration, and combinations thereof.

In some embodiments, the temperature responsive polymers of the present disclosure may be covalently associated with the core particle. In some embodiments, the temperature responsive polymer is poly(N-isopropylacrylamide) (PNIPAM), and the core particle is oxidized carbon black (OCB). In some embodiments, PNIPAM is covalently associated with OCB.

Amphiphilic Polymers

In some embodiments, the core particles of the present disclosure may also be associated with one or more amphiphilic polymers. Amphiphilic polymers generally refer to polymers that include both hydrophilic and hydrophobic moieties. In some embodiments, the phase inversion temperature of the nanoparticles corresponds to the melting point of the hydrophobic moieties of the amphiphilic polymers. In some embodiments, the phase inversion temperature is adjustable as a function of the molecular weight of the hydrophobic moieties of the amphiphilic polymers.

In some embodiments, the amphiphilic polymers comprise block co-polymers. In some embodiments, the hydrophilic moieties in the amphiphilic polymers may include at least one of poly(vinyl alcohol) (PVA), poly(ethylene glycol) (PEG), sorbitol, polysaccharides, polylactone, polyacrylonitrile (PAN), mixtures thereof, and combinations thereof. In some embodiments, the hydrophobic moieties in the amphiphilic polymers may include at least one of polyethylene (PE), poly(vinyl chloride) (PVC), polystyrene (PS), high impact polystyrene (HIPS), polypropylene (PP), polyester, polyacrylonitrile (PAN), mixtures thereof, and combinations thereof.

In some embodiments, the amphiphilic polymers may also include sulfur-based moieties, such as sulfates or sulfonates. In some embodiments, the sulfur-based moieties help inhibit nanoparticle aggregation in the aqueous phase and under high salinities.

In some embodiments, the core particles of the present disclosure may be associated with amphiphilic polymers through non-covalent bonds, such as ionic interactions, acid-base interactions, hydrogen bonding interactions, pi-stacking interactions, van der Waals interactions, adsorption, physisorption, self-assembly, sequestration, and combinations thereof. In some embodiments, the core particles of the present disclosure may be associated with amphiphilic polymers through covalent bonds.

Hydrophilic and Hydrophobic Polymers

In some embodiments, the core particles of the present disclosure may also be associated with hydrophilic polymers, hydrophobic polymers, and combinations of such polymers. In some embodiments, the hydrophilic polymers may include at least one of poly(vinyl alcohol) (PVA), poly(ethylene glycol) (PEG), sorbitol, polysaccharides, polylactone, polyacrylonitrile (PAN), mixtures thereof, and combinations thereof.

In some embodiments, the hydrophobic polymers associated with the core particle may include at least one of polyethylene (PE), poly(vinyl chloride) (PVC), polystyrene (PS), high impact polystyrene (HIPS), polypropylene (PP), polyester, polyacrylonitrile (PAN), mixtures thereof, and combinations thereof.

In some embodiments, the core particles of the present disclosure may be associated with hydrophilic and hydrophobic polymers through non-covalent bonds, such as ionic interactions, acid-base interactions, hydrogen bonding interactions, pi-stacking interactions, van der Waals interactions, adsorption, physisorption, self-assembly, sequestration, and combinations thereof. In some embodiments, the core particles of the present disclosure may be associated with hydrophilic and hydrophobic polymers through covalent bonds.

Magnetic Nanoparticle Preparation

Magnetic nanoparticles of the present disclosure can be prepared by various methods. For instance, in some embodiments, various polymers may be attached to carboxyl-functionalized core particles through ester bond formations. In more specific embodiments, magnetite nanoparticles can be prepared by attaching temperature-responsive polymers to carboxyl-functionalized magnetite nanoparticles via formed ester bonds, amide bonds or carbonate bonds.

In some embodiments, magnetic nanoparticles may be prepared by co-precipitation methods, thermal decomposition methods, and combinations of such methods. In some embodiments, polymers may be attached to core particles through DCC or EDC coupling.

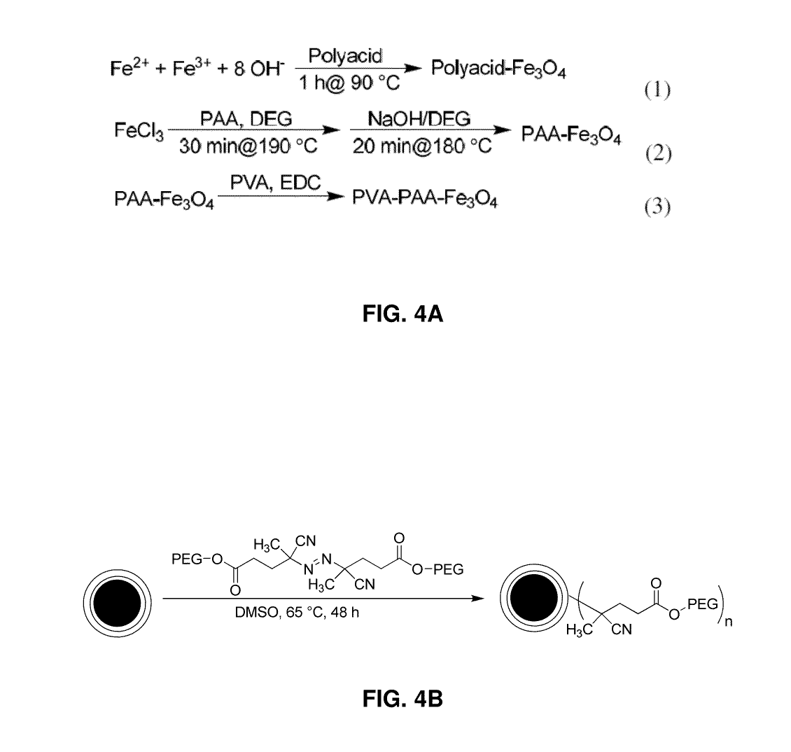

For instance, FIG. 4A provides schemes for the preparation of polyacid-coated magnetite nanoparticles via (1) a co-precipitation method and (2) a thermal decomposition method. The polyacid could be organic acids, such as citric acid or tartaric acid or PAA (poly(acrylic acid)). Likewise, FIG. 4B provides a scheme for the synthesis of polymer-functionalized carbon-coated magnetite nanoparticles using macro polymer initiators. Additional methods of preparing magnetic nanoparticles can also be envisioned.

Hydrocarbon Detection

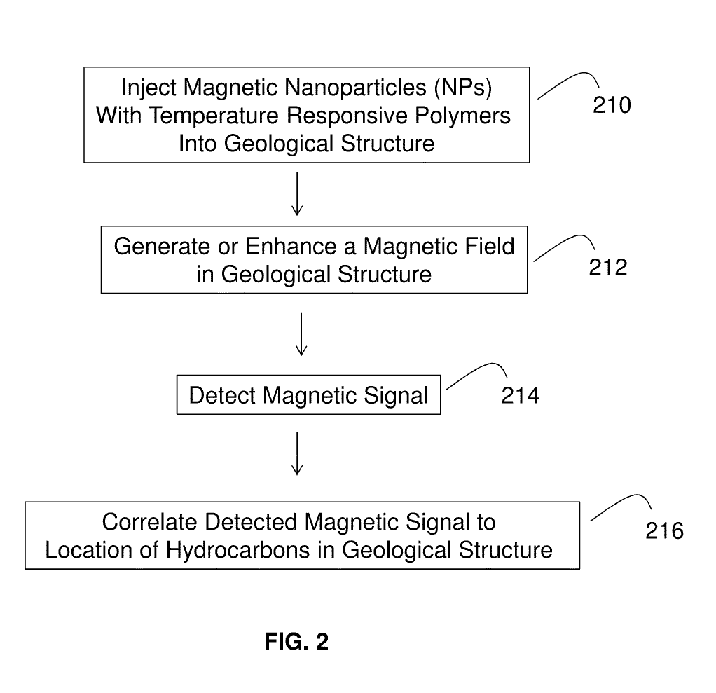

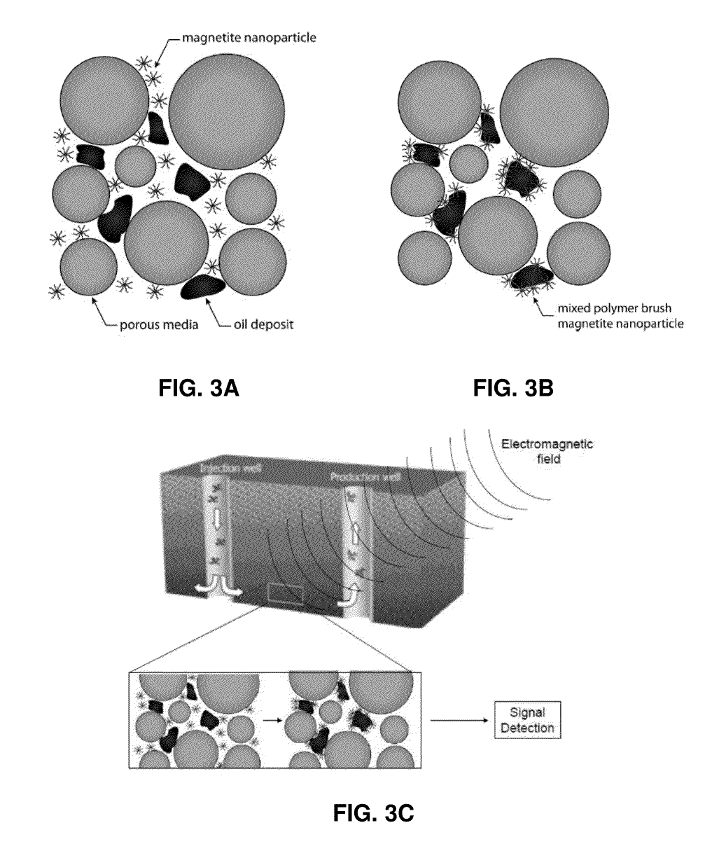

Further embodiments of the present disclosure pertain to systems and methods of magnetically detecting hydrocarbons in a geological structure through the use of the magnetic nanoparticles of the present disclosure. As illustrated in the scheme in FIG. 2 and the diagram in FIG. 3, such systems and methods generally include: injecting magnetic nanoparticles of the present disclosure into the geological structure (step 210); generating or enhancing a magnetic field in the geological structure (step 212); detecting a magnetic signal (step 214); and correlating the detected magnetic signal to location of hydrocarbons in the geological structure (step 216).

Without being bound by theory, it is envisioned that magnetic signals are generated as the magnetic nanoparticles migrate into an organic phase of a fluid (e.g., oil phase) or congregate at an aqueous/organic interface of a fluid (e.g., oil/water interface) in a geological structure. Such migration can thereby highlight the hydrocarbon (e.g., oil) location though the enhanced or generated magnetic field at that location.

As set forth in more detail herein, the magnetic nanoparticles of the present disclosure may be utilized to detect various types of hydrocarbons from various geological structures, especially as the nanoparticles migrate into the organic phase of a fluid (e.g., oil phase) or congregate at the aqueous/organic interface of a fluid (oil/water interface) in a geological structure. Furthermore, various systems and methods may be utilized to generate or enhance magnetic fields in the geological structure, detect magnetic signals, and correlate the detected magnetic signals to the location of hydrocarbons in the geological structure.

Geological Structures

Embodiments of the present disclosure may be applied to various geological structures. In some embodiments, the geological structures may include a downhole environment, such as an oil well or a subterranean formation. In some embodiments, the geological structures of the present disclosure may be associated with various types of rocks, such as sandstone, dolomite, calcite, neutral formations, cationic formations, anionic formations, clays, shale, and combinations thereof.

In some embodiments, the geological structures pertaining to embodiments of the present disclosure may be penetrated by at least one vertical well. In some embodiments, the geological structures of the present disclosure may be penetrated by at least one horizontal well. In some embodiments, the geological structures of the present disclosure may be penetrated by at least one vertical well and at least one horizontal well.

In some embodiments, the geological structure is a reservoir. In some embodiments, the reservoir may be a sub-surface formation, such as an oil well. In some embodiments, the reservoir may be penetrated by at least one vertical well. In some embodiments, the reservoir may be penetrated by at least one horizontal well. In some embodiments, various well-bore angles between horizontal wells and vertical wells may be utilized.

Hydrocarbons

The geological structures of the present disclosure may be associated with various types of detectable hydrocarbons. In some embodiments, the hydrocarbons may be associated with oil deposits. In some embodiments, the hydrocarbons may be derived from petroleum sources. In some embodiments, the hydrocarbons may be crude oil. Additional hydrocarbon sources can also be envisioned.

Nanoparticle Injection

Various systems and methods may also be utilized to inject nanoparticles into geological structures. In some embodiments, the injection may occur by pumping the nanoparticles into a geological structure. In some embodiments, the injection may occur by physically pouring the nanoparticles into a geological structure.

In some embodiments, the nanoparticles of the present disclosure may be dispersed in a fluid prior to injection into a geological structure. In some embodiments, the fluid may include at least one of water, brine, proppant, drilling mud, fracturing fluid, and combinations thereof. In some embodiments, the nanoparticles may be injected into a geological structure while dispersed in a substantially aqueous medium (i.e., >50% water). In other embodiments, the nanoparticles may be injected into a geological structure while dispersed in a substantially organic medium (i.e., >50% organic solvent).

In some embodiments, the nanoparticles may be injected into a geological structure while dispersed in an emulsion, such as an oil in water emulsion, where water is the continuous phase. In some embodiments, the nanoparticles may be injected into a geological structure while dispersed in an invert emulsion, such as a water in oil emulsion, where oil is the continuous phase.

Magnetic Field Generation or Enhancement Various systems and methods may also be used to generate or enhance magnetic fields in geological structures. In some embodiments, such systems and methods generate a magnetic field. In some embodiments, such systems and methods enhance an existing magnetic field. In some embodiments, such systems and methods generate a magnetic field and enhance a magnetic field.

In some embodiments, the magnetic field is generated or enhanced by a magnetic probe in proximity to the geological structure. In some embodiments, magnetic fields can be supplied by permanent magnets, electromagnets, superconducting magnets, solenoids, antennas and combinations thereof. In various embodiments, the magnetic fields may be generated or enhanced by a DC field, an AC field, a radio frequency (RF) field, a microwave field, a pulsed field, or a field that varies in both time and amplitude. In some embodiments, the magnetic probe field may be modulated in a manner to enable frequency-domain, time-domain or phase-shift detection methods to maximize signal-to-noise ratio, and to maximize rejection of natural background noise and 1/f noise. In some embodiments, the source of the electromagnetic field can be from above ground or below ground, such as from an injection well bore, production well bore, monitoring well bore, other well bores, and combinations thereof.

Magnetic Signal Detection

Various systems and methods may also be used to detect magnetic signals in geological structures that contain the magnetic nanoparticles of the present disclosure. In some embodiments, magnetic signals may be detected by at least one of electronic measurements, conductivity measurements, permeability measurements, permittivity measurements, electromagnetic imaging, and combinations thereof.

In some embodiments, magnetic signals may be detected at one or more detection points away from a magnetic probe providing the applied magnetic field. In some embodiments, magnetic signal detection may occur on the surface of a geological structure, or within the geological structure. In some embodiments, magnetic signal detection may be accomplished with a single detector or an array of detectors.

In some embodiments, magnetic signal detectors may be stationary or movable to record magnetic flux data at more than one point. In some embodiments, magnetic signals may be detected with at least one detector that is movable. In some embodiments, the detecting step includes detecting a magnetic signal, moving the at least one detector, and repeating the detecting step to collect magnetic flux data at more than one point.

In some embodiments, detector arrays may be used to detect magnetic signals at a number of points simultaneously. In some embodiments, a magnetic signal detector may be, for example, a superconducting quantum interference device (SQUID) detector or a conventional solenoid, each of which may be fixed or movable over a surface of a reservoir. In some embodiments, magnetic signal detection may be conducted with at least one SQUID detector. In some embodiments, magnetic signal detection may include measuring a resonant frequency in a magnetic probe.

Correlation of Detected Magnetic Signal to Hydrocarbon Location

Various systems and methods may also be used to correlate detected magnetic signals in geological structures to the location of hydrocarbons in the geological structure. In some embodiments, the correlation may occur by the illumination of the magnetic nanoparticles that are in contact with hydrocarbons. In some embodiments, illumination can be due to enhancement of a detectable magnetic signal due to higher local concentration of the magnetic nanoparticles. In some embodiments, magnetic nanoparticles that are in contact with hydrocarbons are illuminated as a result of the generated magnetic field. The illumination can then be utilized to detect the location of the hydrocarbons in a geological structure.

Applications and Advantages

The systems and methods of the present disclosure can be used to more effectively detect the presence of hydrocarbons in various geological structures for numerous purposes. For instance, the systems and methods of the present disclosure can be used in downhole oil detection, enhanced oil recovery, and environmental remediation of organic-contaminated land. In some embodiments, the systems and methods of the present disclosure can be used to provide an effective assessment of stranded downhole oil content within various geological formations. In further embodiments, the systems and methods of the present disclosure can provide a quantitative analysis of the hydrocarbon content in downhole rock formations associated with older oilfields. In further embodiments, the systems and methods of the present disclosure may be used for imaging, such as imaging based on magnetic permeability. In some embodiments, the systems and methods of the present disclosure may be used to enhance a detection signal in response to the presence of oil at a reservoir. In some embodiments, the magnetic nanoparticles of the present disclosure could be used as smart contrast agents for magnetically illuminating the residual oil regions in the porous media and guide the existing techniques in further improving the oil recovery.

### Additional Embodiments

Reference will now be made to various embodiments of the present disclosure and experimental results that provide support for such embodiments. However, Applicants note that the disclosure herein is for illustrative purposes only and is not intended to limit the scope of the claimed subject matter in any way.

### Example 1

### Agglomeration of PNIPAM-OCB at the Aqueous/Organic Interface

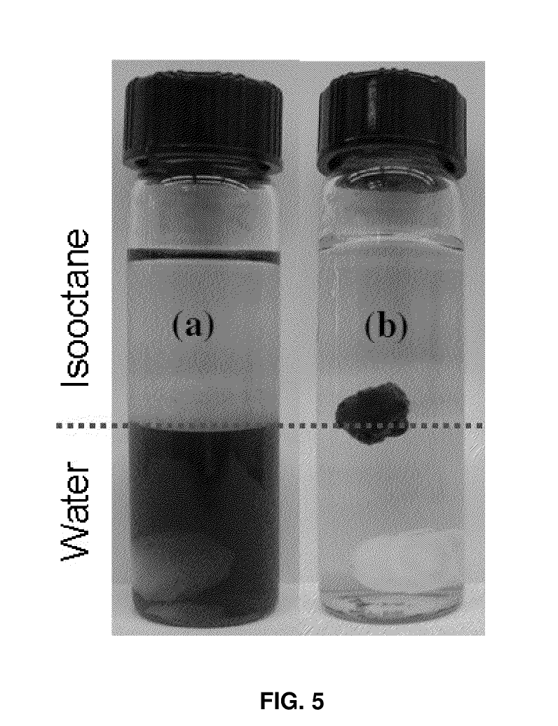

Poly(N-isopropylacrylamide)-functionalized oxidized carbon black nanoparticles (PNIPAM-OCB) were dispersed in synthetic sea brine at room temperature. The synthetic sea brine contained water and isooctane. As illustrated in FIG. 5, vial (a) shows the PNIPAM-OCB nanoparticles were dispersed in the aqueous phase (i.e., water) at room temperature. However, vial (b) shows the PNIPAM-OCB nanoparticles agglomerated at the aqueous/organic interface (i.e., water-isooctane interface) after being heated at 80° C. for 15 minutes.

### Example 2

### Preparation and Characterization of Carbon Onions

This example provides protocols and data relating to the synthesis and characterization of graphene-covered metal nanoparticles (referred to herein as “carbon onions”)

Synthesis of Carbon Onions

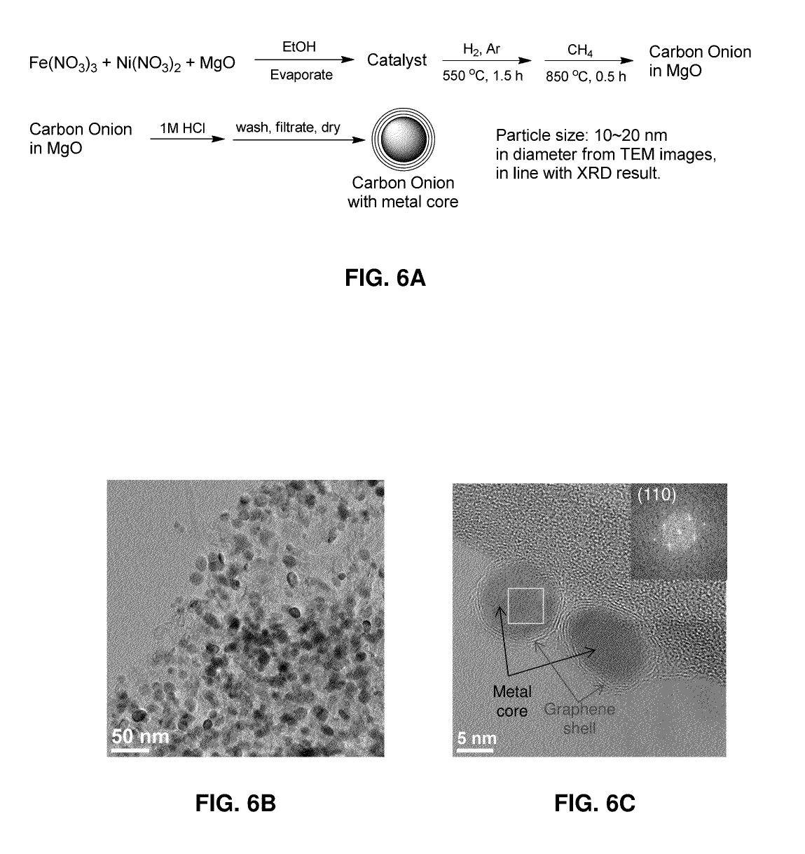

As illustrated in the scheme in FIG. 6A, 1.81 g of Fe(NO₃)₃.9H₂O and 1.24 g of Ni(NO₃)₂.6H₂O were dissolved in 70 mL anhydrous ethanol in a 100 mL beaker. 4.5 g of MgO (325 mesh) was added to solution and sonicated for 30 min to disperse MgO well in solution. Next, the mixture was stirred at 60° C. for 12 h to evaporate the ethanol, thereby producing a yellow powder that serves as the catalyst for synthesizing the carbon onion.

0.5 g of the yellow powder was placed on a quartz boat sitting in a quartz tube. The quartz tube was flushed with Ar flow at 100 cm³ STP min⁻¹ and H₂ flow at 100 cm³ STP min⁻¹ for 10 min under vacuum to remove the air inside the system. Next, the pressure was increased to 1 atm. The yellow powder was annealed at 550° C. for 1.5 h under Ar flow at 100 cm³ STP min⁻¹ and H₂ flow at 100 cm³ STP min⁻¹ before the temperature was increased to 850° C. Then the mixture was heated at 850° C. for 0.5 h at CH₄ flow at 60 cm³ STP min⁻¹ to grow graphene layers on the surface of Fe/Ni. The mixture was then cooled down to room temperature slowly under Ar flow at 100 cm³ STP min⁻¹ and H₂ flow at 100 cm³ STP min⁻¹, producing black powder. The black powder was washed with 50 mL of 1M HCl (three times), 50 mL of 0.1 M HCl (3 times), 50 mL of H₂O (5 times) and 50 mL of acetone (3 times) and dried under vacuum (102 ton) at 25° C. for 12 h.

Preparation of PEI-Functionalized Carbon Onions

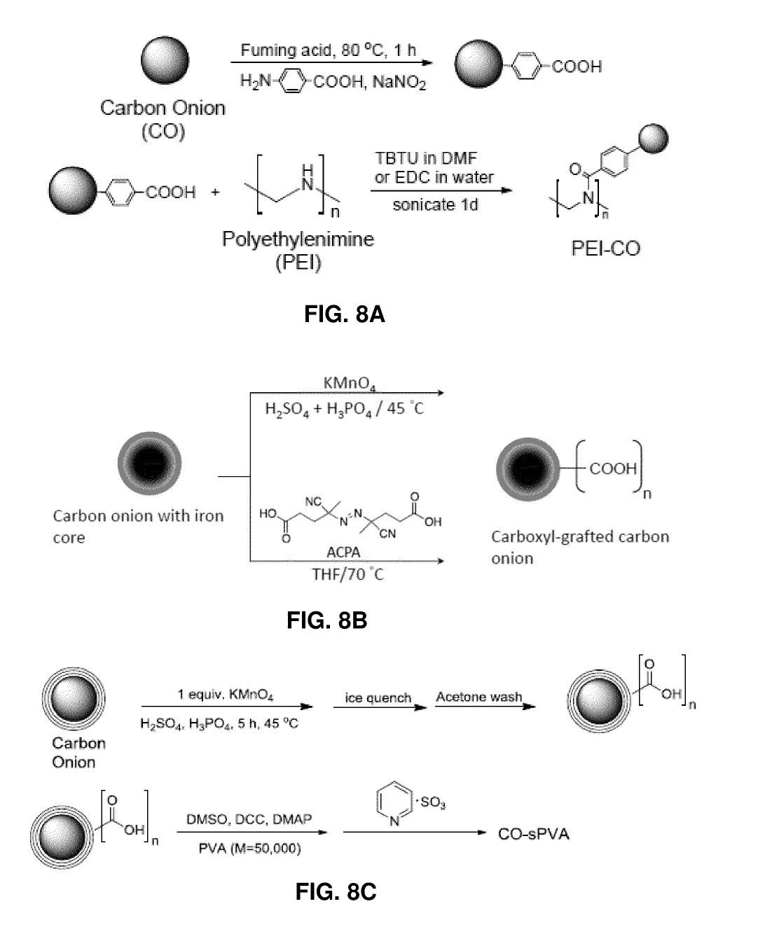

A scheme for the functionalization of carbon onions with polyethyleneimines (PEI) is shown in FIG. 8A.

Preparation of Oxidized Carbon Onions

As illustrated in FIG. 8B, 20 mg of carbon onion, 20 mg of KMnO₄, 9 mL of H₂SO₄, and 1 mL of H₃PO₄ were stirred at 45° C. for 5 h. The nanoparticles were then washed by 10 mL of 0.1 M HCl (3 times), 10 mL of H₂O (3 times), 10 mL of acetone (3 times) and dried under vacuum. The product yield was 22 mg.

Preparation of PVA-Functionalized and Sulfated Carbon Onions

As illustrated in FIG. 8C, PVA-functionalized and sulfated carbon onions were prepared by using pyridine sulfur trioxide as a sulfation reagent to react with PVA grafted carbon onions (CO). The product was then dialyzed to dispense of the unreacted PVA.

Characterization of Carbon Onions

The formed carbon onion was characterized using high resolution transmission electron microscopy (TEM). As shown in the TEM image in FIG. 6B, the size of the carbon onion is about 10 nm. As shown in the TEM image in FIG. 6C, there are three to four layers of graphene on the metal core. In this example, the metal core is the mixture of Fe and Ni.

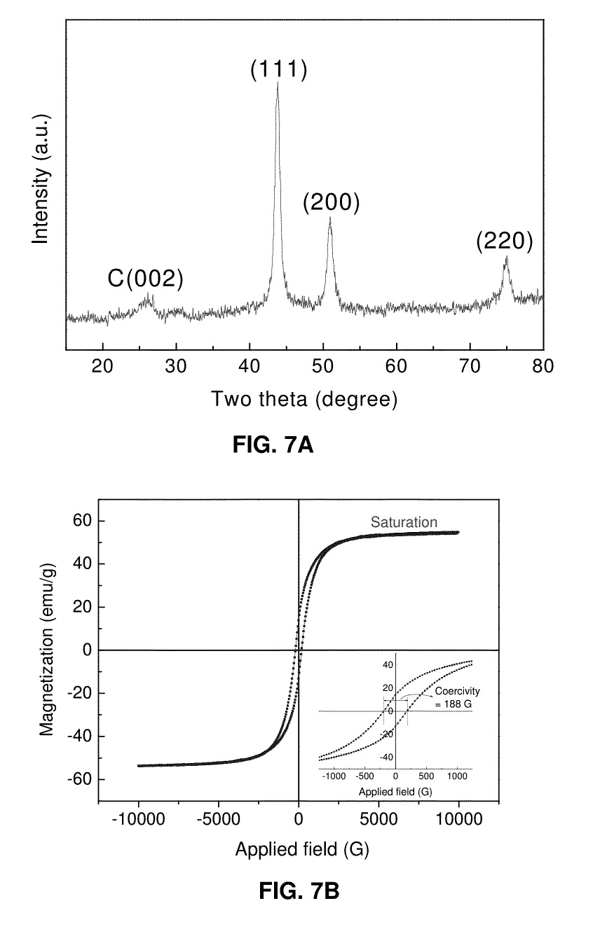

The carbon onions were also characterized by using X-ray diffraction. As illustrated in FIG. 7A, X ray diffraction patterns indicate the fcc structure of FeNi. The X-ray diffraction pattern also confirms that the size of the nanoparticle is 10 nm, which is in accordance with the TEM results shown in FIGS. 6B-C.

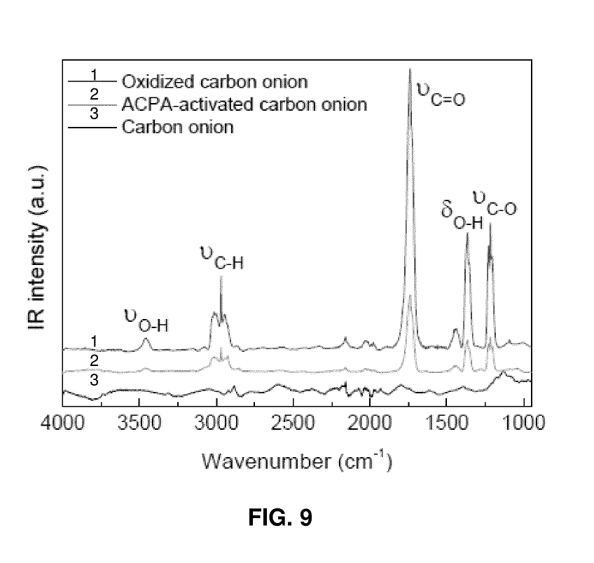

Magnetic property tests summarized in FIG. 7B show that the carbon onions have low coercivity, high saturation magnetization, high susceptibility, and large permeability. The results indicate that carbon onions have optimal magnetic properties. FIG. 9 provides additional data relating to the characterization of various types of carbon onions.

The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1-16. (canceled)

17. A method for magnetically detecting hydrocarbons in a geological structure, wherein the method comprises:
injecting magnetic nanoparticles into the geological structure, wherein the magnetic nanoparticles comprise:
a core particle; and
a temperature responsive polymer associated with the core particle,
wherein the temperature responsive polymer is selected from the group consisting of polyacrylamides, polyalcohols, polyethylene glycols, and combinations thereof,
wherein the temperature responsive polymer facilitates an agglomeration of the magnetic nanoparticles in a fluid at an organic/aqueous interface of the fluid, an organic phase of the fluid, or combinations thereof, and
wherein the agglomeration occurs at a specific temperature or temperature range;
generating or enhancing a magnetic field in the geological structure;
detecting a magnetic signal; and
correlating the detected magnetic signal to locations of hydrocarbons in the geological structure as a function of the agglomeration of the magnetic nanoparticles in the fluid at the organic/aqueous interface of the fluid, the organic phase of the fluid, or combinations thereof.

18. The method of claim 17, wherein the geological structure is an oil or gas reservoir.

19. The method of claim 18, wherein the hydrocarbons comprise crude oil.

20-21. (canceled)

22. The method of claim 17, wherein the magnetic nanoparticles in contact with hydrocarbons are illuminated as a result of the generated or enhanced magnetic field.

23. The method of claim 17, wherein the organic/aqueous interface is a water/oil interface in the geological structure.

24. The method of claim 17, wherein the core particle is selected from the group consisting of magnetite nanoparticles, metal oxide nanoparticles, iron oxide nanoparticles, mixed iron oxide and metal oxide nanoparticles, iron nanoparticles, carbon black, functionalized carbon black, oxidized carbon black, carboxyl functionalized carbon black, carbon nanotubes, functionalized carbon nanotubes, graphenes, graphene oxides, graphene nanoribbons, graphene oxide nanoribbons, metal nanoparticles, silica nanoparticles, silicon nanoparticles, silicon oxide nanoparticles, silicon nanoparticles bearing a surface oxide, and combinations thereof.

25. The method of claim 17, wherein the temperature responsive polymer is selected from the group consisting of poly(N-isopropylacrylamide), N-isopropylacrylamide, polyethylene-b-poly(ethylene glycol), and combinations thereof.

26. The method of claim 17, wherein the core particle is selected from the group consisting of oxidized carbon black, a carbon-coated magnetite nanoparticle, and a graphene-covered metal nanoparticle.

27. The method of claim 17, wherein the temperature-responsive polymer comprises copolymers of N-isopropylacrylamide and polyethylene-b-poly(ethylene glycol).

28. The method of claim 17, wherein the temperature responsive polymer is poly(N-isopropylacrylamide) (PNIPAM), wherein the core particle is oxidized carbon black (OCB), and wherein said PNIPAM is covalently associated with said OCB.

29. The method of claim 17, further comprising amphiphilic polymers associated with the core particle, wherein the amphiphilic polymers comprise both hydrophilic and hydrophobic moieties.

30. The method of claim 29, wherein the hydrophilic moieties are selected from the group consisting of poly(vinyl alcohol) (PVA), poly(ethylene glycol) (PEG), sorbitol, polysaccharides, polylactone, polyacrylonitrile (PAN), mixtures thereof, and combinations thereof.

31. The method of claim 29, wherein the hydrophobic moieties are selected from the group consisting of polyethylene (PE), poly(vinyl chloride) (PVC), polystyrene (PS), high impact polystyrene (HIPS), polypropylene (PP), polyester, polyacrylonitrile (PAN), mixtures thereof, and combinations thereof.

32. The method of claim 17, further comprising hydrophilic polymers associated with the core particle, wherein the hydrophilic polymers are selected from the group consisting of poly(vinyl alcohol) (PVA), poly(ethylene glycol) (PEG), sorbitol, polysaccharides, polylactone, polyacrylonitrile (PAN), mixtures thereof, and combinations thereof.

33. The method of claim 17, further comprising hydrophobic polymers associated with the core particle, wherein the hydrophobic polymers are selected from the group consisting of polyethylene (PE), poly(vinyl chloride) (PVC), polystyrene (PS), high impact polystyrene (HIPS), polypropylene (PP), polyester, polyacrylonitrile (PAN), mixtures thereof, and combinations thereof.

34. A system for magnetically detecting hydrocarbons in a geological structure, wherein the system comprises:
a pump suitable for injecting magnetic nanoparticles into the geological structure, wherein the magnetic nanoparticles comprise:
a core particle; and
a temperature responsive polymer associated with the core particle,
wherein the temperature responsive polymer is selected from the group consisting of polyacrylamides, polyalcohols, polyethylene glycols, and combinations thereof,
wherein the temperature responsive polymer facilitates an agglomeration of the magnetic nanoparticles in a fluid at an organic/aqueous interface of the fluid, an organic phase of the fluid, or combinations thereof, and
wherein the agglomeration occurs at a specific temperature or temperature range;
an apparatus suitable for generating or enhancing a magnetic field in the geological structure where the magnetic nanoparticles have been injected;
an apparatus suitable for detecting a magnetic signal resulting from an illumination of the magnetic nanoparticles agglomerated at the organic/aqueous interface of the fluid, the organic phase of the fluid, or combinations thereof; and
an apparatus suitable for correlating the detected magnetic signal to locations of hydrocarbons in the geological structure as a function of the agglomeration of the magnetic nanoparticles in the fluid at the organic/aqueous interface of the fluid, the organic phase of the fluid, or combinations thereof.

35. The system of claim 34, wherein the temperature responsive polymer is selected from the group consisting of poly(N-isopropylacrylamide), N-isopropylacrylamide, polyethylene-b-poly(ethylene glycol), and combinations thereof.

36. The system of claim 34, wherein the core particle is selected from the group consisting of oxidized carbon black, a carbon-coated magnetite nanoparticle, and a graphene-covered metal nanoparticle.

37. The system of claim 34, wherein the temperature-responsive polymer comprises copolymers of N-isopropylacrylamide and polyethylene-b-poly(ethylene glycol).

38. The system of claim 34, wherein the temperature responsive polymer is poly(N-isopropylacrylamide) (PNIPAM), wherein the core particle is oxidized carbon black (OCB), and wherein said PNIPAM is covalently associated with said OCB.
